(19) United States
(12) Patent Application Publication
(10) Pub. No.: US 2023/0154606 A1
(43) Pub. Date: May 18, 2023

(54) REAL-TIME MEDICAL DEVICE TRACKING METHOD FROM ECHOCARDIOGRAPHIC IMAGES FOR REMOTE HOLOGRAPHIC PROCTORING

(71) Applicant: ARTINESS S.R.L., Milano (IT)

(72) Inventors: Omar PAPPALARDO, Milano (IT); Filippo PIATTI, Milano (IT); Giovanni ROSSINI, Milano (IT); Jacopo MARULLO, Milano (IT); Stefano PITTALIS, Milano (IT)

(21) Appl. No.: 17/917,496

(22) PCT Filed: Apr. 1, 2021
(86) PCT No.: PCT/IB2021/052728

§ 371 Date: Oct. 6, 2022

(30) Foreign Application Priority Data
Apr. 6, 2020 (IT) 102020000007252

(51) Int. Cl. G16H 40/67; G06T 11/00; G06T 7/73; G16H 30/40

(52) U.S. Cl. CPC G16H 40/67; G06T 11/00; G06T 7/73; G16H 30/40; G06T 2210/41; G06T 2207/30048

(57) ABSTRACT

A method for visualizing, by a remote holographic device, a medical image stream includes streaming a medical image stream as obtained from a medical image acquisition apparatus to a virtual machine on a server, the medical image stream including digital representation of a medical device lacking physical tracking markers or hardware tracking systems and inserted into a body organ, identifying digital position and orientation of the medical device and at least two pre-defined digital anatomical landmarks on at least one subset of images in the medical image stream, generating a graphical element representing the digital position and orientation of the medical device, and overlaying the graphical element to the at least one subset of images, obtaining an overlaid image stream, reformatting the overlaid image stream into a video signal, and sending the video signal to the remote holographic device for visualization.

The present invention concerns a real-time medical device tracking method from echocardiographic images for remote holographic proctoring.

### STATE OF THE ART

According to the American Academy of Family Physicians (AAFP), proctoring is an objective evaluation of a physician's clinical competence by a proctor who represents, and is responsible to, the medical staff. New medical staff members seeking privileges or existing medical staff members requesting new or expanded privileges are proctored while providing the services or performing the procedure for which privileges are requested. In most instances, a proctor acts only as a monitor to evaluate the technical and cognitive skills of another physician. A proctor does not directly provide patient care, has no physician-patient relationship with the patient being treated, and does not receive a fee from the patient.

The terms proctorship and preceptorship are sometimes used interchangeably. However, a preceptorship is different in that it is an educational program in which a preceptor teaches another physician new skills and the preceptor has primary responsibility for the patient's care.

There are three types of proctoring: prospective, concurrent, and retrospective. In prospective proctoring, prior to treatment, the proctor either reviews the patient personally or reviews the patient's chart. This type of proctoring may be used if the indications for a particular procedure are difficult to determine or if the procedure is particularly risky. In concurrent proctoring, the proctor observes the applicant's work in person. This type of proctoring usually is used for invasive procedures so that the proctor can give the medical staff a firsthand account to assure them of the applicant's competence. Retrospective proctoring involves a retrospective review of patient charts by the proctor. Retrospective review is usually adequate for proctoring of noninvasive procedures.

Concurrent proctoring is time consuming and more difficult in organization, but it can be the most valuable.

Document US2019339525A1 discloses an interventional procedure can be performed less invasively with live 3D holographic guidance and navigation, which overcomes some inconveniences of visualization on 2D flat panel screens. The live 3D holographic guidance can provide a complete holographic view of a portion of the patient's body to enable navigation of a tracked interventional instrument/device. However, in order to realize this, the disclosed system uses an optical generator and tracking devices, which can be reflective markers and can be optically tracked to provide the tracking data. Such markers are therefore physical devices which renders the interventional operation dependent on the these devices. Applying these devices to already existing interventional tools can be difficult or even impossible. Integrating these devices into new interventional tools can be expensive and can increase the tool's size as well as have in impact on its functionality.

A strong need is felt to use telecommunication technologies to allow remote virtual proctoring, which would make it possible to use the best experts in the world to proctor physicians of a hospital. In this regard, a need is felt to have a method that tracks the interventional tool during the operation without any physical marker device to be added to the tracked instrument, i.e. by image computation only. In this way, the development of the interventional instruments and the development of tracking and proctoring techniques are made independent with all the benefits that this brings about, including cost savings, dedicated researches, size reduction, increase in tracking speed leading to actual real-time remote assistance, and avoiding malfunctioning of the physical markers.

### Object and Subject-Matter of the Invention

The object of the present invention is to provide a real-time medical device tracking method during a surgical intervention for remote holographic proctoring.

The subject of the present invention is a real-time medical device tracking method according to the attached claims.

It is also specific subject of the present invention a server which is configured to be used in the invention method, attached to the attached server claims.

### DETAILED DESCRIPTION OF INVENTION EMBODIMENTS

### List of Figures

The invention will now be described for illustrative but not limitative purposes, with particular reference to the drawings of the attached figures, in which:

FIG. 1 shows the general proctoring assistance concept of the invention;

FIG. 2 shows a detailed flow chart of an embodiment according to the invention;

FIG. 3 shows a simplified diagram of the doctor and the proctor using the invention;

FIGS. 4 to 6 show various training sets used for training the AI in the invention method applied to a heart intervention;

FIG. 7 shows a UNET neural network loss trend in the training dataset (dark grey) and in the validation dataset (light grey), in an example of neural network training in the method according to the invention;

FIG. 8 shows an example of invention neural network results on a validation image according to the invention. The first row relates to device segmentation, while the second one to the heart's leaflets. The left images show the neural network segmentation overlapped to the cropped diagnostic images, the central ones show the segmentation provided by a test provider and in the right ones the neural network segmentations are shown; and

FIG. 9 shows an example of neural network results, according to the invention, in which the leaflets segmentation (second row) is wrong and incomplete, while the device segmentation (first row) is accurate.

It is specified here that elements of different embodiments can be combined together to provide further embodiments without limits respecting the technical concept of the invention, as the skilled person understand directly and unambiguously from what will be described.

The present description also refers to the prior art for its implementation, with regard to the detailed characteristics not described, such as for example elements of lesser importance usually used in the prior art in solutions of the same type.

When an element is introduced, it is always meant that it can be “at least one” or “one or more”.

When a list of elements or features is listed in this description, it is understood that the finding according to the invention “comprises” or alternatively “is composed of” such elements.

### Embodiments

Although the following embodiments are referred to proctoring during heart interventions, it is to be understood that the invention method enables remote proctoring of any surgical or medical intervention of any organ or group of organs.

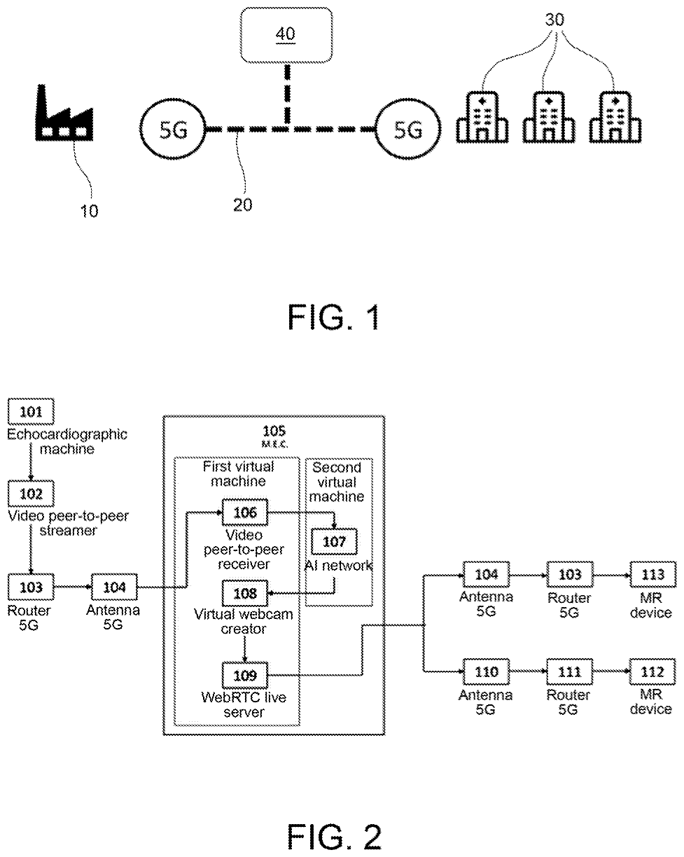

In fact, referring to FIG. 1, a medical device company 10 offering the proctoring service uses the telecommunication network 20 to connect to one or more hospitals 30, wherein the data from the interventions at the hospitals are transferred preferably using a Multi-access edge computing (MEC) 40.

Although the following embodiments refer to the use of 5G telecommunication network, it is to be understood that the method of the invention can well be realized by other current or future types of network, including cabled connection and Wi-Fi, with different times of latency of course.

According to an aspect of the invention, echocardiographic images of the patient hearts valves and heart structures are acquired during a transcatheter surgical procedure while implanting a cardiovascular medical device. Any type of medical device, including those that are used to operate and not to be implanted can be tracked according to the invention.

In this regard, the medical device needs not having a physical marker (or any tracking hardware system or component) in/on it, in order to be tracked by the invention method. The invention method works solely by image processing. However, a medical device with one or more physical markers could be used to complement in another way the invention method in some circumstances.

At the moment of the acquisition, the images contains the patient anatomical structures of interest (e.g., heart valve leaflets, annulus, left atrium and left ventricle) and the medical device that is maneuvered by the operator.

Making reference to the flow chart of FIG. 2, a live imaging of patient's heart is taken by an echocardiographic machine 101 in the operating theater. Such live imaging can be captured through a video capture system (e.g. HDMI converter) and can be transmitted as raw data to a streaming software 102 (e.g. video peer-to-peer streaming) on a local computer (in the operating theater), preferably preserving the same resolution and frame rate of 101 output.

The streaming software on 102 gets the video input and generates a streaming connection (e.g. the User Datagram Protocol (UDP)) pointing to the IP address of the virtual machine (e.g. Windows operating system, which today is better suited for connection between Mixed Reality devices) inside the server 105 (wherein e.g. the M.E.C. environment is implemented), in which the streaming software receiver 106 is located.

The video peer-to-peer receiver 106 receives the streaming signal through a 5G router 103 and a 5G antenna 104, preferably preserving the same resolution and frame rate of 101 output. At this point, according to a preferred embodiment of the invention, a data transfer of nearly 20 Mbit/s is generated from 102 to 106.

In general, according to the invention, a 5G router can be connected via LAN or WiFi cable to a video streamer and via 5G radio signal to a 5G antenna. Moreover, according to an aspect of the invention, in place of the router a computer with a 5G SIM could be used to have a direct access to the network.

According to another embodiment, the router can be integrated into the end-user holographic (visualization) device (in FIG. 2, block 103 is integrated into block 113). More in general, the end-user holographic (visualization) device can be configured to connect to the (5G) network.

The video streaming is then passed, preferably as a continuous stream of images, to the AI network 107, which is trained to recognize on the echocardiographic images the position of the above medical device and at least two anatomical landmarks (mitral valve leaflets to annulus insertion in the example) for at least a subset of the stream images, preferably for every image processed (i.e., for every video frame). The anatomical landmarks can be defined by one or more of the following: position, orientation, shape, specific points or representative points. The landmarks can have a twofold effect: they can help the proctored people (when represented by a graphical elements overlaid onto the image stream according to an aspect of the invention) or the doctor to recognize a region of interest, and they can be used to create a 3D representation of the operation, as explained below.

Each frame can be converted to a grayscale image, in order to be consistent with the dataset used during the AI training phase. This operation is computed in a highly parallel manner, taking advantage of data level parallelism (SIMD).

Although a medical device is here mentioned, the invention method may enable more than one medical devices used in an intervention to be recognized concurrently.

The received frames can be cached in a local buffer, i.e. a small set of frames, and then removed from the local buffer as soon as the AI processes the individual images. In case the AI is processing the frames slower than the speed of the buffer filling, the cache may become completely full; in this case, it is preferable not to stop the video stream, but to use instead the original video frame, without the information from the AI, in order to guarantee a smooth frame flow back to the users.

According to an aspect of the invention, the AI network 107 (or, more in general, an expert algorithm) generates graphical elements to be overlaid to each processed image (e.g. lines or segments of any shape) for the representation of the device position (and preferably orientation as well) and the anatomical landmarks. This computation can be carried out by exploiting the high level of parallelism offered by the graphics processing unit, in order to ensure that the operation has the lowest delay. The AI network produces an output, which is a (e.g. continuous) stream of images that are advantageously reformatted into a video with the same format of the input one, which is then passed directly to a virtual video creator 108. Preferably, the virtual video creator is a virtual webcam creator, preparing the video stream as if it were generated by a live camera. The AI network can only send the list of coordinates of those pixels that must be highlighted on a given echocardiographic image. This can be done to reduce the amount of data exchanged between the two VMs. Less data exchanged means a reduction of the latency of 1/10 compared to sending the entire post-processed image directly.

The virtual video creator receives the pixel coordinate bytes, intelligently processes them together with the initial full-frame image pixels data to produce the final overlaid output video stream. In a preferred realization, this operation has been executed grouping every frame in batches of a meaningful size, to further enhance the speed of the process. In the case of a continuous stream of images, the invention can make use of a buffering system that stores the images in a queue before the super-imposition process ends, waiting for the AI to send its response. The virtual video creator processes each frame exploiting the power of every computation unit of the VM by using advanced parallel computation algorithms. The invention can scale horizontally by using the full computational power of MEC in case of multiple participants (e.g. hospitals) connected together.

The AI 107 is preferably hosted in a second virtual machine (e.g. with Linux operating system because it performs better today) that can be hosted on the same layer of the M.E.C. as for the first virtual machine. The virtual webcam creator 108 is hosted on the first virtual machine with the video peer-to-peer receiver 106. The communication between the two VMs makes use of a real-time data exchange technology. The data are exchanged between the two virtual machines completely in RAM, through the use of an in-memory database, ensuring a ping time smaller than 2 milliseconds.

The virtual webcam creator 108 may encode the input from 107 as virtual live webcam video signal, preferably preserving the same resolution and frame rate. This video encoding process optimizes the high throughput coming originally from the echocardiographic machine 101 to be subsequently exploited with a streaming protocol virtual server 109. Preferably, the virtual server 109 is a WebRTC virtual server establishing multi-peer connections with connected users by exploiting the WebRTC transmission protocol and thus reducing to 1/10 the total amount of data network transmission (e.g. to 2 Mbit/s) with respect to other technologies.

The streaming protocol 109 lies on the first virtual machine and reads the virtual webcam signal of 108 as a video chat system (only if it deals with a webcam signal, otherwise the streaming protocol does not read the signal of 108 as a chat) and process it to send binary data to the end-user holographic devices 112 and/or 113 through a 5G antenna 110 and a 5G router 111 and/or 5G antenna 104 and 5G router 103 respectively.

Although in the present description the device 112 is a physical device to be used by a human surge, the invention equally applies when the device 112 is a virtual device integrated in a robot, which is configured to control the medical device. Therefore, in the present application a physical and virtual device of visualization are equally intended when describing and claiming the invention.

This binary data contains the information of each processed pixel of the video. According to an embodiment of the invention, this information is received by 112 and 113 at the same moment and, in the case of the current technology, it is applied to change the texture material properties of an holographic 3D cube representing a virtual monitor, showing exactly the video output of the virtual server 109, i.e. the echocardiographic images with the medical device and possibly anatomical landmarks recognition (including corresponding graphical elements, see below).

Advantageously, 112 is located to a remote location (worn by the doctor) distant from 101, while 113 is located in the same location of 101. Nonetheless, the whole system allows to the two operators (112—proctor, 113—surgeon) to share the same holographic echocardiography visualization with AI device tracking at the same exact moment in time. Using a 5G network, the delay can be less than 0.5 seconds with respect to the output of 101.

The whole system may rely on the M.E.C. environment on server 105, which is a technology that allows hosting both the network connection with 5G routers and antenna, and the virtual machines working as a dedicated cloud computing service.

The M.E.C. infrastructure is implemented to be a decentralized edge-computing point close to the data source, i.e., 101—and the hospital facilities. This decentralization of the processing computing is for the time being unique to M.E.C. infrastructures, and allows to have computing resources closer to the data source than any other network system (e.g. 4G) would make it possible. The use of 5G technology will be then advantageous to obtain very low latency in data transmission to and from the M.E.C even in presence of high bandwidth of data transmission and real-time connections. In particular, low latency is guaranteed also in case of multiple connections, i.e. a high number of connected users. This can occur in two situations:

1) During support by 112 to 113, N>50 participants connect to spectate the work of 112 and 113 with a learning purpose;

2) 105 hosts in parallel N>20 pairs of virtual machines that concurrently manage N connections hospital-proctor. All these connections may pass through a set of antennas 104, 110 closer to the computing servers.

### Concurrent Visualization

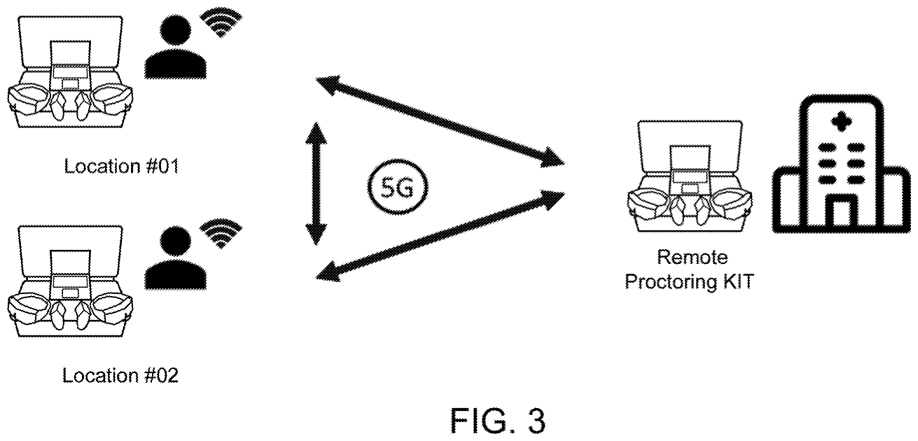

Making reference to FIG. 3, the invention system allows using a remote proctoring kit at the hospital site while proctoring happens at a different location. For example, location #1 and location #2 can be remote, enabling for a double proctoring. In this case, the two visualization locations may communicate with each other through a telecommunication network, which can be the same telecommunication network used for remote visualization.

The 3D echo-machine acquires the echocardiography and passes it to a local computer that manages to visualize the video on a local and remote Hololens device. In order to do this, the video is first sent to a MEC server. The mixed reality video is then sent back to a local antenna and then to the Hololens, as well as to a remote receiver and then to the remote Hololens.

### Mixed Reality

The holographic echocardiography visualization above described can be in mixed reality, according to a specific embodiment of the invention. In this case, a 3D anatomy model of the heart (or other organ) is prepared beforehand (for each patient based on some scan) and superposed to the live streaming. Moreover, the AI recognizes not only the position and orientation of the medical device, but also anatomical landmarks.

In this way, the medical device can be visualized within the anatomy model, so that the doctor can decide to move the object differently. The anatomical landmarks can also serve for other clinical purposes.

The holographic visualization device can be for example HoloLens, Magic leap, Lenovo Explorer, or any other, be it holographic or not. Moreover, the mixed reality can include any other useful element such as a button panel.

The superposition of the echography image onto the 3D model may be effected by a rigid transformation. However, if the anatomical part is moving (e.g. beating heart) then the rigid superposition is not possible. In this case, a rigid affinity transformation can be used.

For example, in the echocardiographic acquisition of the mitral valve we can identify the saddle horn (p₁=[x₁y₁z₁]) [Netter, Frank H. Atlas Of Human Anatomy. Philadelphia, Pa.: Saunders/Elsevier, 2011], the mid-point of the posterior annulus (p₂=[x₂y₂z₂]) [Netter, Frank H. Atlas Of Human Anatomy. Philadelphia, Pa.: Saunders/Elsevier, 2011] and the apex of the ventricle (p₃=[x₃y₃z₃]) [Netter, Frank H. Atlas Of Human Anatomy. Philadelphia, Pa.: Saunders/Elsevier, 2011]. In this example of course we are taking single point of an area which in most cases is sufficient.

After the identification of the 3 corresponding markers (P₁=[X₁ Y₁ Z₁], P₂=[X₂ Y₂ Z₂], P₃=[X₃ Y₃ Z₃]) on the 3D pre-operative model, to super-impose the 2D image over the 3D model, the system to be solved is the following:

$$[ \begin{matrix}{X}_{1} & {X}_{2} & {X}_{3} \\ {Y}_{1} & {Y}_{2} & {Y}_{3} \\ {Z}_{1} & {Z}_{2} & {Z}_{3} \\ 1 & 1 & 1\end{matrix} ] = {STSHR}_{x} {R}_{y} {R}_{z} [ \begin{matrix}{x}_{1} & {x}_{2} & {x}_{3} \\ {y}_{1} & {y}_{2} & {y}_{3} \\ {z}_{1} & {z}_{2} & {z}_{3} \\ 1 & 1 & 1\end{matrix} ]$$

$$Translate ( T ) = [ \begin{matrix}1 & 0 & 0 & Δ x \\ 0 & 1 & 0 & Δ y \\ 0 & 0 & 1 & Δ z \\ 0 & 0 & 0 & 1\end{matrix} ]$$

$$Δ x , Δ y , Δ z are , respectively , the displacemente in the x , y , z direction$$

$$Scale ( S ) = [ \begin{matrix}{S}_{x} & 0 & 0 & 0 \\ 0 & {S}_{y} & 0 & 0 \\ 0 & 0 & {S}_{z} & 0 \\ 0 & 0 & 0 & 1\end{matrix} ]$$

$${S}_{x} , {S}_{y} , {S}_{z} are , respectively , the scale in the x , y , z direction$$

$$Shear ( SH ) = [ \begin{matrix}1 & h \text{?} & h \text{?} & 0 \\ h \text{?} & 1 & h \text{?} & 0 \\ h \text{?} & h \text{?} & 1 & 0 \\ 0 & 0 & 0 & 1\end{matrix} ]$$

$$h \text{?} is the shear factor in two different directions$$

$$Rotation about the x axis ( R \text{?} ) = [ \begin{matrix}1 & 0 & 0 & 0 \\ 0 & cos θ \text{?} & - sin θ \text{?} & 0 \\ 0 & sin θ \text{?} & cos θ \text{?} & 0 \\ 0 & 0 & 0 & 1\end{matrix} ]$$

$$Rotation about the y axis ( {R}_{y} ) = [ \begin{matrix}cos {θ}_{y} & 0 & sin {θ}_{y} & 0 \\ 0 & 1 & 0 & 0 \\ - sin {θ}_{y} & 0 & cos {θ}_{y} & 0 \\ 0 & 0 & 0 & 1\end{matrix} ]$$

$$Rotation about the \text{?} axis ( R \text{?} ) = [ \begin{matrix}cos θ \text{?} & - sin θ \text{?} & 0 & 0 \\ sin θ \text{?} & cos θ \text{?} & 0 & 0 \\ 0 & 0 & 1 & 0 \\ 0 & 0 & 0 & 1\end{matrix} ]$$

$${θ}_{x} , θ \text{?} , θ \text{?} are , respectively , the rotation angles in the x , y , z direction$$

$$\text{?} \text{indicates text missing or illegible when filed}$$

Moreover, the 3D anatomical model can be dynamical, i.e. a series of model frames, wherein the model body organ has different shape at different frames (at least for a subset of model frames). When the same holds for the acquired image stream (and therefore for the image stream with overlaid graphical elements), the superposition of the stream onto the model can be a problem. In this case, the recognition of the correct acquired body organ frame to be superimposed to a given model frame can be performed by identifying the acquired (overlaid) frame for which the error of the affine transformation to the given model frame is minimum. This can be realized by mathematical transformation or by a trained algorithm. Of course, this can be done only for some of the model frames and interpolation or other methods can be used in between.

### Training of Neural Network

The landmark to be recognized by the AI are decided beforehand. Therefore, AI is to be trained to recognize image by image until it finds the reperes.

The reperes can be areas, therefore in this case positioning on the model should be decided. Since this would change the precision of positioning the medical device, according to an aspect of the invention the AI can be trained to make the superposition optimized by using more than three reperes.

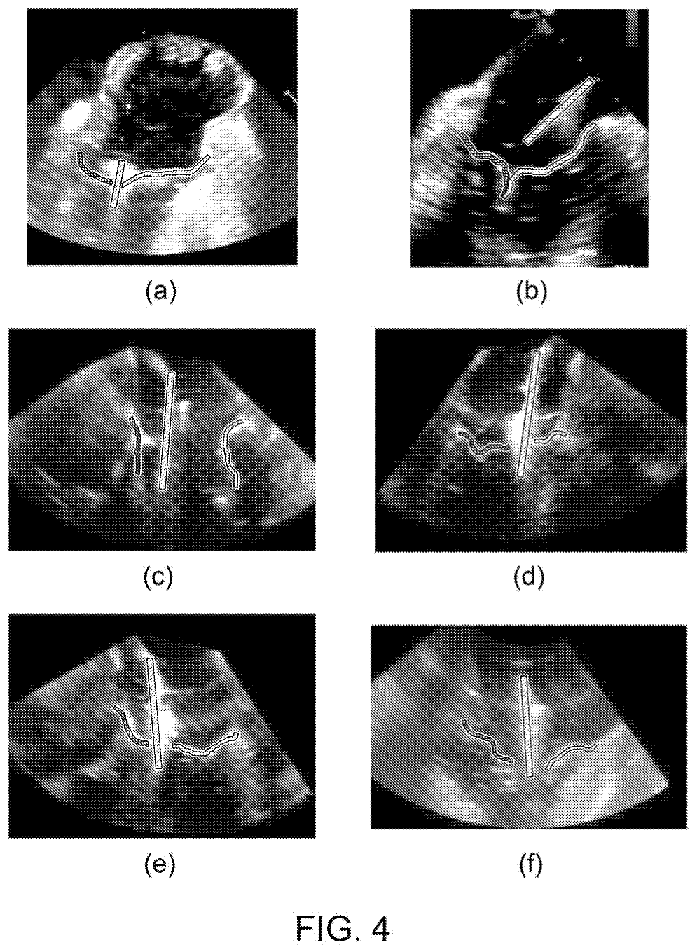

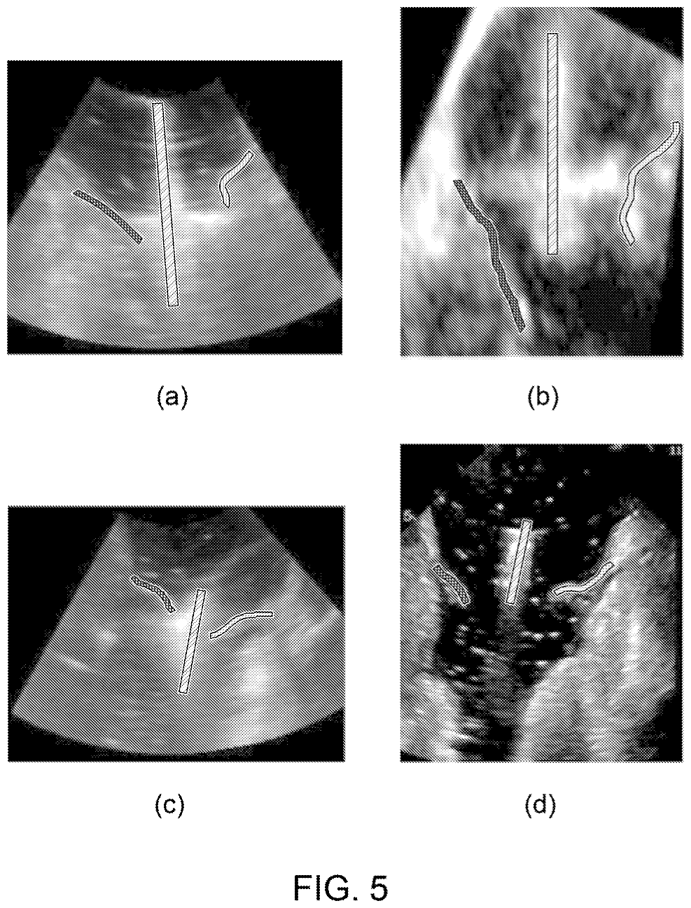

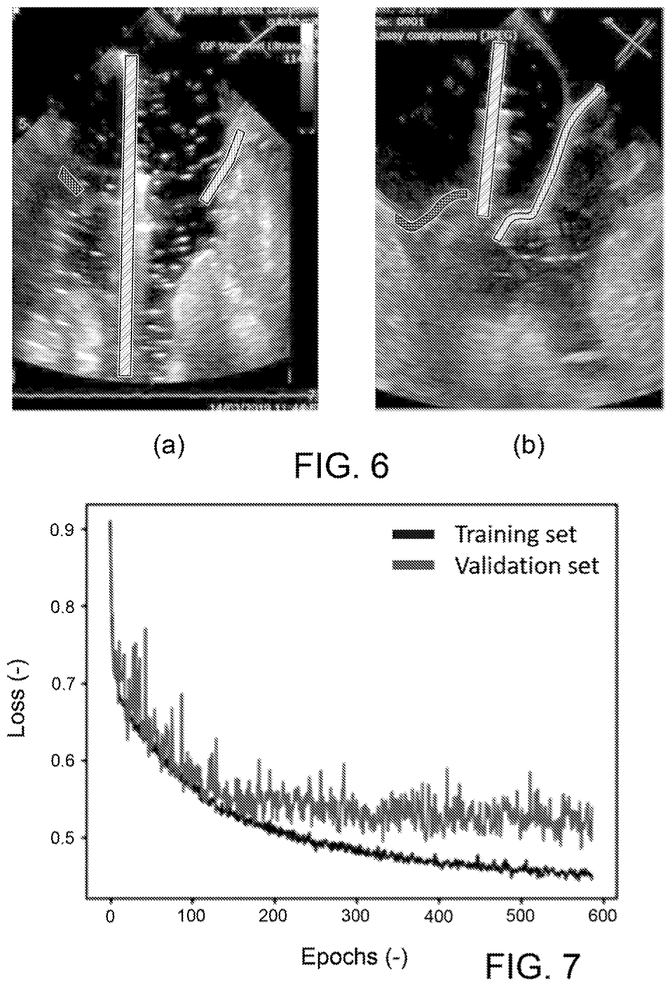

FIGS. 4-6 show exemplary training sets with two reperes (square grid patterned segment for heart valve leaflet and cross-patterned segment for the other heart valve leaflet) and a medical device (oblique line patterned segment).

### Specific Example of Invention AI (Expert Algorithm)

### The Exemplary Situation

An AI-based system was developed to identify an Abbott Mitraclip™ valve repair device for suturing the cardiac valve flaps in videos acquired as temporal sequences of 2D echocardiographic views.

In particular:
Each frame of the video is analyzed to segment the device;
The outcome of the model is the binary segmentation of the Mitraclip™ in each frame.

### Dataset Description and Preprocessing

Making reference to FIGS. 4 to 6, images saved during echocardiography executions and the corresponding annotations constitute the basic starting point. In particular, for each image, the annotations shows the Mitraclip™ as an oblique line patterned segment, while the mitral valve leaflets are identified as square grid patterned and cross patterned segments.

Images were acquired during echocardiography performed in 3D or 2D mode and with two different manufacturers' probes. In particular:
186 images from 2D echocardiography with a GE probe;
823 images from 2D echocardiography with a Philips probe;
166 images from 3D echocardiography with a GE probe;
216 images from 3D echocardiography with a Philips probe.

During a first test, only images from 2D echocardiography with the Philips probe have been be used, but the development of the model exploited all types of images in order to increase the dataset size.

The training, validation and test set were randomly built (random choice among images), with a fixed seed, but with the constraint of including all the images from the same echocardiography acquisition in the same set. Approximatively 10% of the entire available dataset was included in the test set, 10% in the validation dataset and the remaining 80% in the training set.

To identify the device in the mask construction (the mask is a twin image superposed to the original image, in the twin image the segmented areas are present) the oblique line pattern pixels are selected, while the square grid and cross pattern ones are considered to extract the leaflets. The mask construction starting from the annotation allows you to include or not include the mitral leaflets. In the first case the mask is 3D, including two channels referred to the mitral leaflets (in general, each channel may correspond to a segmented object), while in the second one is two-dimensional.

### Neural Network Structure

Since the goal is to segment a two-dimensional image (more in general, a N-dimensional image, for example a 3D-image, e.g. from echography or fluoroscopy or other imaging system), we have chosen to use a UNET neural network, starting from the typical model available at https://github.com/milesial/Pytorch-UNet/blob/master/unet/u net_model.py. We chose to train the model by minimizing the complementary value of the dice score.

The neural network may have one or two output classes, depending on whether the tester wants to identify only the device or even the mitral leaflets. If both the leaflets and the device are to be segmented, the losses of the two output channels are averaged. It was included also the possibility to train the model with dropout (i.e. without the presence of leaflets and medical device).

Each batch includes 16 images and the learning rate and weight decay were initialized respectively to 1e-3 and 1e-4. The learning rate was updated every 800 epochs, with a 0.618 gamma.

We applied several transformations to the images before using them as neural network inputs. More in details, the training set images were:
randomly rotated with a range of 90° degrees;
randomly cropped to be square, with the side equal to the minimum size found in the training set images;
randomly flipped;
randomly changed in their brightness, if desired;
resized to have a size of 128 by 128.

The validation dataset images were only centrally cropped with the side equal to the minimum size found in the training set images and then they were resized to 128 by 128.

### Neural Network Performances

We trained the NN model using different configurations:

1. classical 2D UNET (link above);

2. 2D UNET with dropout;

3. 2D UNET with dropout and leaflets segmentation;

4. 2D UNET with dropout, leaflets segmentation and brightness transformation.

The performances were evaluated through the analysis of the Mitraclip™ segmentation in the validation set images and some tests performed by the test provider on new videos. The best performances were reached in the third case above: the leaflets segmentation proved to be a helpful auxiliary task in improving device segmentation results. In fact, recognizing the areas where the leaflets are present reduces the search area for the medical device. Moreover, it defines more constraints in the reciprocal position between leaflets and device. The graph in FIG. 7 shows the loss trend in the training (dark grey) and in the validation dataset (light grey).

In fact, recognizing the areas where there are leaflets establishes constraints also in the search for the medical device, considering the reciprocal positions. In general, by adding an auxiliary task, the network loss evaluates the performance on multiple tasks (loss=primary_loss+auxiliary_loss, where the primary task here is the segmentation of the device). We therefore obtain a model that can perform multiple tasks and consequently the risk of overfitting on a specific task (i.e. the primary one) is reduced and the model generalizes better. Many layers and therefore weights of the network are shared between the various tasks, even if they are then followed by specific layers for each task. Sharing the layers makes the training more constrained, as the weighted sum of the two partial losses is minimized. Greater correctness in the shared levels will then be exploited by the specific layers for the primary activity, i.e. for the segmentation of the device. In a method where the whole recognition is made digitally, and deals with surgical operations, the accuracy of the recognition is critical. The recognition of the (at least) two anatomical landmarks can be then useful to superpose the overlaid image stream onto a pre-defined 3D model of the body organ, as above explained. The importance of recognition can therefore be twofold.

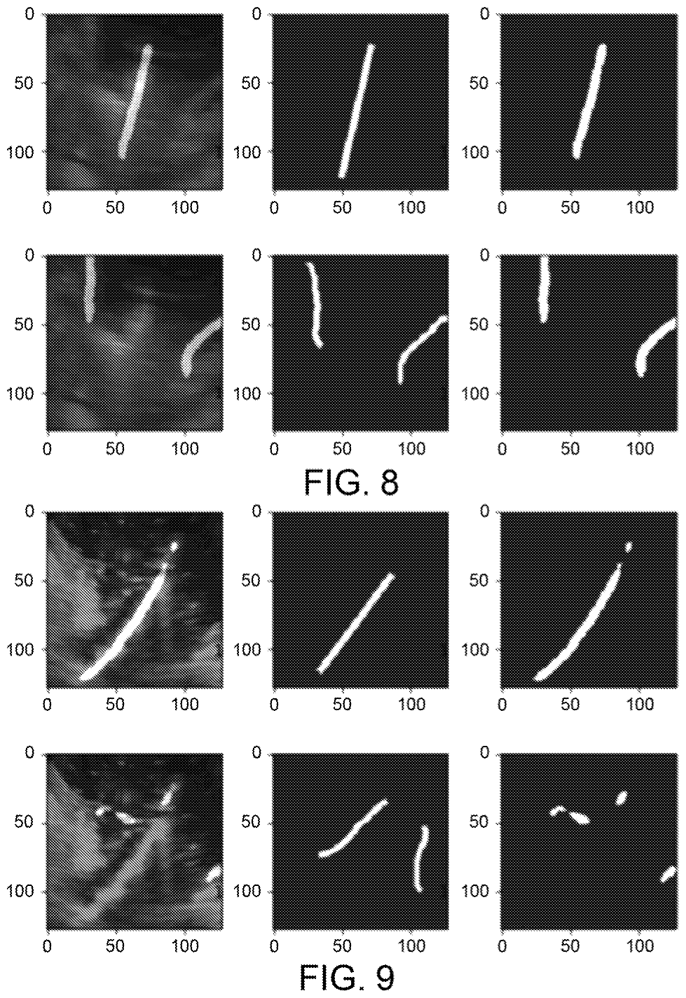

FIG. 8 shows an example of results obtained on validation images. The top row in the image relates to the Mitraclip™ segmentation, while the bottom row relates to the leaflets segmentations. In both rows, the image on the left shows the neural network prediction overlapped to the cropped diagnostic image, the central image shows the original segmentation by the test provider and in the right one only the neural network prediction is shown.

In general, device segmentation is better than that of the leaflets, which in some cases is wrong or incomplete, as we can see in the FIG. 9. The neural network Mitraclip™ segmentation seems to be accurate and it adapts to the shape of the device better than the linear approximation provided by the test provider.

### Model Application to Echocardiography Videos

The model (expert algorithm) was used to identify the Mitraclip™ in videos acquired by performing echocardiography: the videos are temporal sequences of 2D echocardiographic views.

Once acquired the video, it is split in its frames. Each of them is given as input to the neural network and the prediction is done. The results on the different frames are then grouped in sequence and they are saved in an mp4 video.

In order to improve the rendering of the video, we decided to add the possibility to extend the prediction for each frame to n previous and subsequent frames with an increasing opacity factor: this post-processing allows for a more uniform segmentation and a more stable display of the video itself.

### Advantages of the Invention

The invention technology allows remote virtual proctoring, which would make it possible to use the best experts in the world to proctor physicians of a hospital.

It also enables a proctor to perform in rapid sequence a series of proctoring in different and reciprocally distant areas, which could not be reached rapidly: this increases the covering of hospitals by the same proctor.

Moreover, it allows an “emergency” intervention, or an intervention, which was not planned as a proctorship intervention. This can be particularly advantageous when unexpected complications occur on more or less standard cases, for which the intervention of the proctor a priori was not foreseen.

In the foregoing, the preferred embodiments have been described and variants of the present invention have been suggested, but it is to be understood that those skilled in the art will be able to make modifications and changes without thereby departing from the corresponding scope of protection, as defined by the claims attached.

1-16. (canceled)

17. A method for visualizing, by a remote holographic device, a medical image stream, the method comprising the following steps:
A) streaming a medical image stream as obtained from a medical image acquisition apparatus to a virtual machine on a server, the medical image stream comprising a digital representation of a medical device lacking physical tracking markers or hardware tracking systems and inserted into a body organ;
B) identifying, by an expert algorithm on the sole basis of the medical image stream, running on said virtual machine, digital position and orientation of the medical device and at least two pre-defined digital anatomical landmarks on at least one subset of images in the medical image stream;
C) generating a graphical element representing the digital position and orientation of the medical device, and overlaying the graphical element to said at least one subset of images, obtaining an overlaid image stream;
D) reformatting the overlaid image stream into a video signal; and
E) sending the video signal to the remote holographic device for visualization.

18. The method of claim 17, wherein in step C) at least two further graphical elements representing the at least two pre-defined digital anatomical landmarks are generated and overlaid to said at least one subset of images.

19. The method of claim 17, wherein in step A) the medical image stream consists of echocardiographic images and the at least two pre-defined digital anatomical landmarks are mitral valve leaflets to annulus insertion.

20. The method of claim 19, wherein the echocardiographic images represent a transcatheter surgical procedure.

21. The method of claim 17, wherein streaming the medical image stream is performed by generating a User Datagram Protocol streaming connection pointing to the IP address of the virtual machine.

22. The method of claim 17, wherein the overlaid graphical element is a segment.

23. The method of claim 17, wherein between step D) and step E) the video signal is encoded in such a way to optimize a throughput compatible with WebRTC streaming protocol.

24. The method of claim 17, wherein in step A) the virtual machine is in a Multi-access Edge Computing environment.

25. The method of claim 17, wherein steps C) and D) are performed on a virtual machine that is different from the virtual machine performing step B).

26. The method of claim 17, wherein the following step is performed concurrently with step E):
F) sending the video signal to a local holographic device for holographic visualization at an intervention site.

27. The method of claim 17, wherein the server is a 5G server.

28. The method of claim 17, wherein in step D) the video signal is further processed by a virtual webcam creator.

29. The method of claim 17, wherein in step C) the at least two identified pre-defined digital anatomical landmarks are used to superpose the overlaid image stream onto a pre-defined 3D model of a body organ.

30. The method of claim 29, wherein if in step A) the medical image stream is constituted by a series of patient body organ image frames, wherein a shape of the body organ differs for at least a subset of body organ image frames, and the pre-defined 3D model of the body organ is constituted by a series of model frames, the following steps are executed to superpose the overlaid image stream onto the pre-defined 3D model:
S1) for each model frame, calculating a rigid affinity transformation of at least a subset of frames of the overlaid image stream into the model frame; and
S2) for each model frame, superimposing a specific frame of the overlaid image stream, wherein the specific frame minimizes error in the rigid affinity transformation of step S1).

31. The method of claim 17 wherein the remote holographic device is a virtual device integrated in a robot, which is configured to control the medical device.

32. A server, wherein the server is configured to execute steps B), C), and D) of claim 17.
